(19) United States
(12) Patent Application Publication
(10) Pub. No.: US 2024/0360361 A1
(43) Pub. Date: Oct. 31, 2024

(54) PIPERAZINE BIS-UREA AND BIS-THIOUREA-BASED CORROSION INHIBITOR COMPOUNDS FOR STEEL PROTECTION

(71) Applicant: QATAR UNIVERSITY, Doha (QA)

(72) Inventors: Abdulilah DAWOUD, Doha (QA); Mohanad SHKOOR, Doha (QA); Rem JALAB, Doha (QA); Mazen KHALED, Doha (QA); Mohammed SAAD, Doha (QA); Ibnelwaleed HUSSEIN, Doha (QA)

(21) Appl. No.: 18/647,768

(22) Filed: Apr. 26, 2024

Related U.S. Application Data
(60) Provisional application No. 63/462,316, filed on Apr. 27, 2023.
(60) Provisional application No. 63/462,324, filed on Apr. 27, 2023.

(51) Int. Cl. C09K 15/30; C07D 295/13

(52) U.S. Cl. CPC C09K 15/30; C07D 295/13

(57) ABSTRACT

Described herein are piperazine bis-urea and bis-thiourea based inhibitor compounds, methods of inhibiting steel corrosion in the presence of corrosive media comprising introducing said compounds into the corrosive media in contact with the steel, and a synthesis of said compounds.

This application claims priority to, and the benefit of, U.S. Provisional Patent Application No. 63/462,316 filed Apr. 27, 2023, and U.S. Provisional Patent Application No. 63/462,324 filed Apr. 27, 2023. The entirety of each application is incorporated herein by reference for all purposes.

### FIELD

Described herein are piperazine bis-urea and bis-thiourea based inhibitor compounds, methods of inhibiting steel corrosion in the presence of corrosive media comprising introducing said compounds into the corrosive media in contact with the steel, and a synthesis of said compounds.

### BACKGROUND

Sour crude oil and gas condensate containing H₂S and CO₂ cause corrosion of oil and gas transportation pipelines. Corrosion failures were estimated to cost around 3-4% of the world's GDP annually, reaching about 2.5 trillion USD (B. D. B. Tiu, R. C. Advincula, React. Funct. Polym. 95 (2015) 25-45; S. A. Umoren, M. M. Solomon, V. S. Saji, Corrosion inhibitors for sour oilfield environment (H₂S corrosion), in: V. S. Saji, S. A. Umoren. (Eds.), Corros. Inhib. Oil Gas Ind., First, 2020 Wiley-VCH Verlag GmbH & Co. KGaA, 2020: pp. 229-254). Therefore, preventing metallic corrosion attacks is important for lowering economic losses and as an environmental and safety precaution. However, acidizing and descaling treatments in pipeline cleaning tends to introduces mineral acids. For example, well acidizing treatments rely on injecting corrosive mineral acids, usually HCl (15-28 wt %) to dissolve deposits and enhance flow (C. Verma, et al. Mater. Adv. 2 (2021) 3806-3850). Carbon steel is the most widely employed material for oil and gas transportation pipelines due to its cost efficiency and mechanical durability, but it is particularly susceptible to corrosion and loss of functionality when exposed to corrosive environments and at elevated temperature and pressure conditions.

The injection of corrosion inhibitors (CIs) is one of the most efficient and economical practical mitigation approach. Based on their mode of action and chemical composition, inhibitory chemicals can be categorized into barriers, film-forming, scavengers, and neutralizers. CIs are either organic compounds adsorbing onto the metal surface or inorganic compounds reacting with anodic or cathodic parts. The formation of a protective film layer by organic inhibitors is facilitated by existing electron donor sites, including heteroatoms (N, O), aromatic rings, and polar functional groups. Alcohols (E. Barmatov, et al. Corros. Sci. 123 (2017) 170-181), amines (K. Xhanari, et al. Chem. Pap. 71 (2017) 81-89), triazoles (Z. Rouifi, et al. Chem. Data Collect. 22 (2019) 100242), benzimidazole (I. B. Onyeachu, et al. Corros. Sci. 168 (2020) 108589), and quinoxalines (Olasunkanmi, L. O. et al. J. Phys. Chem. C 2015, 119, 28, 16004-16019) have been shown to possess the properties of efficient inhibitors. For acidizing treatments, commercial inhibitory formulations often comprise quaternary ammonium salts, nitrogen-containing heterocycles, carbonyls, and acetylenic alcohols (M. A. Migahed, et al. Electrochim. Acta. 53 (2008) 2877-2882). However, these formulators are often not environmentally friendly, expensive, and require high concentrations to be efficient (K. Haruna, et al. J. Environ. Chem. Eng. 9 (2021) 104967).

Studies have also shown that piperazine-containing compounds often exhibit low oxidation rates in the inhabitation of Fe and fast adsorption (S. Ghaffari, et al. Prot. Met. Phys. Chem. Surfaces. 55 (2019) 1195-1206). Further, piperazine compounds have the potential to form complexes with metal ions, rendering them efficient chelating agents. For example, a recent study disclosed a quinolin-8-ol piperazine derivative that exhibited a green inhibition efficiency of 95% in 1M HCl (M. El Faydy, et al. J. Mol. Liq. 354 (2022) 118900).

Therefore, what are needed are new inhibitor compounds to tackle the challenge of mitigating steel corrosion during the acidizing treatment of oil and gas wells, which will help to diminish the severity of metallic corrosion in wide industrial applications.

### SUMMARY OF THE INVENTION

In one aspect, provided herein is an inhibitor compound of Formula (I):

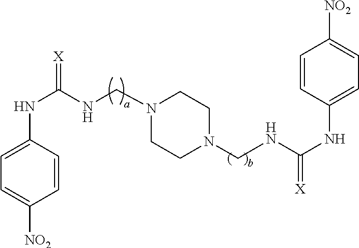

or a salt thereof; wherein X is selected from O and S; and
a and b are independently an integer selected from 2, 3, 4, 5, 6, 7, and 8.

Also provided herein a method of inhibiting steel corrosion in the presence of a corrosive media comprising introducing an inhibitor compound of Formula (II):

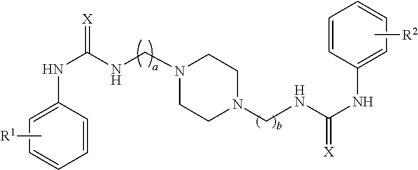

or a salt thereof; wherein
X is selected from O and S;
R¹ and R² are independently selected from NO₂, alkyl, alkoxy, aryl, heteroaryl, and halogen wherein the aryl and heteroaryl are optionally substituted with alkyl, alkoxy, —OH, —COOH, —C(O)NH₂, —NH₂, —NHCOOH, —CN, —NO₂, or halogen; and
a and b are independently an integer selected from 2, 3, 4, 5, 6, 7, and 8;
   or composition thereof into the corrosive media wherein the steel is in contact with the corrosive media. In one embodiment, the corrosive media is an aqueous corrosive media. In one embodiment, the corrosive media comprises an acidic corrosive media, for example hydrochloric acid (HCl). In one embodiment, the concentration of the HCl is between about 0.5 M and about 7 M, between about 1 M and about 5 M, between about 0.5 M and about 2 M, or between about 4 M and about 6 M.

In one embodiment, the concentration of the inhibitor compound of Formula (II) in the corrosive media is between about 10 ppm and about 250 ppm, between about 10 ppm and about 100 ppm, between about 50 ppm and between about 100 ppm, between about 100 ppm and 150 ppm, between about 100 ppm and 200 ppm, or between about 150 ppm and 250 ppm.

In one embodiment, the inhibitor compound of Formula (I) or Formula (II) is Inhibitor 1:

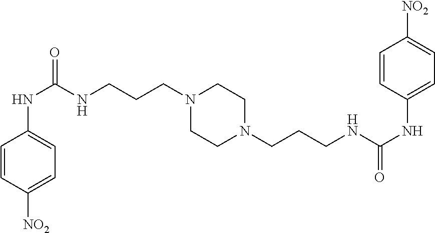 [SMILES: O=C(NCCCN1CCN(CCCNC(=O)Nc2ccc([N+](=O)[O-])cc2)CC1)Nc1ccc([N+](=O)[O-])cc1]

or a salt thereof.

In one embodiment, the inhibitor compound of Formula (I) or Formula (II) is Inhibitor 2:

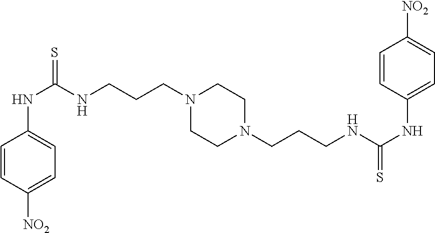 [SMILES: O=[N+]([O-])c1ccc(NC(=S)NCCCN2CCN(CCCNC(=S)Nc3ccc([N+](=O)[O-])cc3)CC2)cc1]

or a salt thereof.

As described herein, it has been surprisingly discovered that both Inhibitor 1 and Inhibitor 2 reduce the corrosion rate of different steel types in acidic environments that mimic the typical conditions of well acidizing treatments in the oil and gas industry. For example, Inhibitor 1 exhibited 97% corrosion inhibition efficiency in 1M HCl at 200 ppm and Inhibitor 2 exhibited 95% corrosion inhibition efficiency at 200 ppm concentration in 5M HCl. Additionally, both compound inhibitors demonstrated safe probabilities of carcinogenicity, eye corrosion and irritation, skin sensitization, biodegradation, and aquatic toxicity in a model to evaluate their eco-toxicities.

Also described herein is a synthesis for the inhibitor compounds of Formula (II-A).

### BRIEF DESCRIPTION OF FIGURES

FIG. 1 are Tafel curves in the presence of Inhibitor 1 at concentrations ranging from 50 ppm to 200 ppm in 1M HCl at a scan rate of 1.0 mV/s.

FIG. 2 are AC impedance curves in the presence of Inhibitor 1 at concentrations ranging from 50 ppm to 200 ppm in 1M HCl.

FIG. 3A is a SEM micrograph of polished C-steel.

FIG. 3B is a SEM micrograph of polished C-steel in 1M HCl in the absence of 200 ppm Inhibitor 1.

FIG. 3C is a SEM micrograph of polished C-steel in 1M HCl in the presence of 200 ppm Inhibitor 1.

FIG. 4 is an image of the three-electrode corrosion cell assembly.

FIG. 5 are AC impedance curves in the presence of Inhibitor 2 at concentrations ranging from 10 ppm to 200 ppm in 5M HCl.

FIG. 6 are Tafel curves in the presence of Inhibitor 2 at concentrations ranging from 10 ppm to 200 ppm in 5M HCl at a scan rate of 1.0 mV/s.

FIG. 7A is a SEM micrograph of polished C-steel.

FIG. 7B is a SEM micrograph of polished C-steel in 5M HCl in the absence of 200 ppm Inhibitor 2.

FIG. 7C is a SEM micrograph of polished C-steel in 5M HCl in the presence of 200 ppm Inhibitor 2.

### DETAILED DESCRIPTION

Described herein are inhibitor compounds of Formula (I), methods of inhibiting steel corrosion in the presence of corrosive media comprising introducing an inhibitor compound of Formula (II) into the corrosive media in contact with the steel, and a synthesis of the inhibitor compounds of Formula (II-A).

In certain embodiments, the inhibitor compound of Formula (I) or Formula (II) is Inhibitor 1:

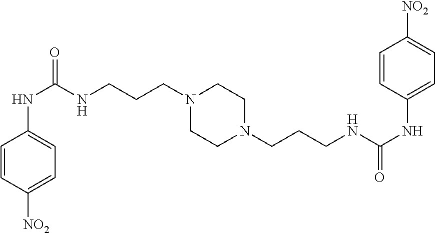 [SMILES: O=C(NCCCN1CCN(CCCNC(=O)Nc2ccc([N+](=O)[O-])cc2)CC1)Nc1ccc([N+](=O)[O-])cc1]

or a salt thereof.

Inhibitor 1 is a soluble organic corrosion inhibitor for use in aqueous corrosion environments to protect steel. As described in Example 2, it exhibits a high efficiency against steel corrosion of 97% at a concentration of 200 ppm and 92% at 100 ppm in 1 M HCl. Further, as described in Example 3, the eco-toxicity of Inhibitor 1 was predicted using the admetSAR web tool, a comprehensive source for investigating chemical ADMET properties that relies on a machine-learning model, and results showed that it is predicted to be non-carcenogenic and eco-friendly with a high biodegradation probability of 75%.

In other embodiments, the inhibitor compound of Formula (I) or Formula (II) is Inhibitor 2:

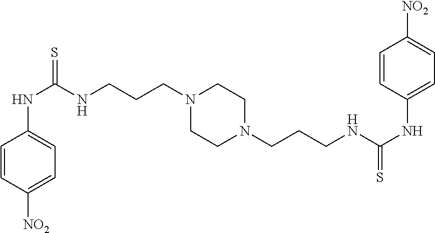 [SMILES: O=[N+]([O-])c1ccc(NC(=S)NCCCN2CCN(CCCNC(=S)Nc3ccc([N+](=O)[O-])cc3)CC2)cc1]

or a salt thereof.

Inhibitor 2 is also a soluble organic corrosion inhibitor for use in aqueous corrosion environments to protect steel. As described herein in Example 5, Inhibitor 2 achieved 95% efficiency at 200 ppm concentration in 5M HCl. The machine-learning model derived for predicting chemical ADMET properties was further employed to evaluate its eco-toxicity, and assessment outcomes revealed that Inhibitor 2 is predicted to be non-toxic and biodegradable with a safe impact in many applications (Example 6).

### Definitions

The terms “a” and “an” do not denote a limitation of quantity, but rather denote the presence of at least one of the referenced item. The term “or” means “and/or”. Recitation of ranges of values merely intend to serve as a shorthand method of referring individually to each separate value falling within the range, unless otherwise indicated herein, and each separate value is incorporated into the specification as if it were individually recited herein. The endpoints of all ranges are included within the range and independently combinable. All processes described herein can be performed in a suitable order unless otherwise indicated herein or otherwise clearly contradicted by context. The use of example, or exemplary language (e.g., “such as”), is intended merely to better illustrate the invention and does not pose a limitation on the scope of the invention on unless otherwise claimed.

“Alkyl” is a branched or straight chain saturated aliphatic hydrocarbon group. In one non-limiting embodiment, the alkyl group contains from about 1 to about 6 carbon atoms, from about 1 to about 4 carbons, or from about 1 to about 3 carbons. Examples of alkyl include, but are not limited to, methyl, ethyl, n-propryl, isopropyl, n-butyl, sec-butyl, t-butyl, n-pentyl, tert-pentyl, neopentyl, and n-hexyl. Alkyl can also include cycloalkyl.

“Alkoxy” is the group —O—R′, wherein R′ is alkyl. Examples of alkoxy include, but are not limited to, methoxy, ethoxy, n-propoxy, isopropoxy, n-butoxy, tert-butoxy, sec-butoxy, n-pentoxy, and n-hexyloxy.

“Aryl” is an aromatic ring system wherein the ring atoms are carbon atoms. Aryl is optionally substituted as described herein and can be monocyclic or polycyclic, e.g., bicyclic or tricyclic. Examples of aryl moieties include, but are not limited to, those having 6 to 20 ring carbon atoms, i.e., C₆₋₂₀ aryl; 6 to 15 ring carbon atoms, i.e., C₆₋₁₅ aryl, and 6 to 10 ring carbon atoms, i.e., C₆₋₁₀ aryl. Examples of aryl moieties include, but are limited to, phenyl and naphthyl.

“Heteroaryl,” is an aromatic ring system wherein one or more (in some or any embodiments, 1, 2, 3, or 4) of the ring atoms is a heteroatom independently selected from O, S(O)o. ₂, NH, and N, and the remaining ring atoms are carbon atoms, and where the ring may be optionally substituted as described herein. The heteroaryl group can be monocyclic or bicyclic. The heteroaryl group is bonded to the rest of the molecule through any atom in the ring system, valency rules permitting.

The term “cyano” refers to —CN.

The term “halo” refers to independently to bromo, chloro, fluoro, or iodo.

The term “nitro” refers to —NO₂.

The term “corrosion inhibitor” refers to a substance(s) that lessens or reduces an amount of, and/or that prevents, retards, slows, hinders, delays the deterioration of a metal surface by oxidation or other chemical reaction. Corrosive substances that can cause corrosion, particularly of metal surfaces used during the production, recovery, transportation, storage and refining of hydrocarbons or various natural gases, include water with high salt contents, acidic inorganic compounds such as inorganic acids, carbon dioxide (CO₂) or hydrogen sulfide (H₂S), natural organic acids, and microorganisms. Preferred corrosion inhibitors of the present invention inhibit the destructive effect such substances have on various metal surfaces.

Corrosion inhibition efficiency (IE % or r %) is a measure of the efficiency of an inhibitor to effectively decrease a corrosion rate. Corrosion inhibition efficiency may be measured with the Tafel extrapolation of potentiodynamic polarization (PDP).

Any compound used in or formed by the processes described herein may be modified to make an inorganic or organic acid or base addition salt thereof to form a salt, if appropriate and desired. The salts of the present compounds can be prepared from a parent compound that contains a basic or acidic moiety by chemical processes. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Examples of salts include, but are not limited to, mineral or organic acid salts of basic residues such as amines; alkali or organic salts of acidic residues such as carboxylic acids; and the like. The salts include the salts and the quaternary ammonium salts of the parent compound formed, for example, from inorganic or organic acids that are not unduly toxic. For example, acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like, or using a different acid that produces the same counterion.

### Compound Inhibitors of Formula (I)

In one embodiment of Formula (I), X is S. In one embodiment of Formula (I), X is O. In one embodiment of Formula (I), including any of the foregoing, a is 2. In one embodiment of Formula (I), including any of the foregoing, a is 3. In one embodiment of Formula (I), including any of the foregoing, a is 4. In one embodiment of Formula (I), including any of the foregoing, a is 5. In one embodiment of Formula (I), including any of the foregoing, a is 6. In one embodiment of Formula (I), including any of the foregoing, a is 7. In one embodiment of Formula (I), including any of the foregoing, a is 8. In one embodiment of Formula (I), including any of the foregoing, b is 2. In one embodiment of Formula (I), including any of the foregoing, b is 3. In one embodiment of Formula (I), including any of the foregoing, b is 4. In one embodiment of Formula (I), including any of the foregoing, b is 5. In one embodiment of Formula (I), including any of the foregoing, b is 6. In one embodiment of Formula (I), including any of the foregoing, b is 7. In one embodiment of Formula (I), including any of the foregoing, b is 8. In one embodiment of Formula (I), including any of the foregoing, both a and b are 2. In one embodiment of Formula (I), including any of the foregoing, both a and b are 3. In one embodiment of Formula (I), including any of the foregoing, both a and b are 4. In one embodiment of Formula (I), including any of the foregoing, both a and b are 5. In one embodiment of Formula (I), including any of the foregoing, both a and b are 6. In one embodiment of Formula (I), including any of the foregoing, both a and b are 7. In one embodiment of Formula (I), including any of the foregoing, both a and b are 8.

In certain embodiments, the inhibitor compound of Formula (I) is formulated in corrosion protection formulations and coatings. In certain embodiments, the inhibitor compound of Formula (I) is further modified to protect against corrosion under extreme conditions, which would further benefit the oil and gas industry.

### Methods of Inhibiting Steel Corrosion

Also provided herein a method of inhibiting steel corrosion in the presence of a corrosive media comprising introducing an inhibitor compound of Formula (II):

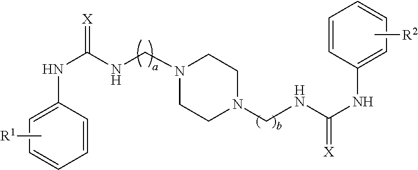

or a salt thereof; wherein
X is selected from O and S;
R¹ and R² are independently selected from NO₂, alkyl, aryl, alkoxy, heteroaryl, and halogen wherein the aryl and heteroaryl are optionally substituted with alkyl, alkoxy, —OH, —COOH, —C(O)NH₂, —NH₂, —NHCOOH, —CN, —NO₂, or halogen; and
a and b are independently an integer selected from 2, 3, 4, 5, 6, 7, and 8;
   or a composition thereof into the corrosive media wherein the steel is in contact with the corrosive media. In one embodiment, the corrosive media is an aqueous corrosive media. In one embodiment, the corrosive media comprises an acidic corrosive media. The acid may be in liquid or gas form. In one embodiment, the corrosive media has a pH between 0 and about 7, between about 0 and about 5, or between 0 and about 3. In some embodiments, the corrosive media comprises at least one acid, including but not limited to, hydrochloric acid, sulfuric acid, nitric acid, hydrofluoric acid, phosphoric acid, acetic acid, citric acid, and formic acid. In one embodiment, the corrosive media comprises hydrochloric acid. In another embodiment, the corrosive media comprises an acidic gas, such as carbon dioxide (CO₂) or hydrogen sulfide (H₂S).

In one embodiment, including any of the foregoing, the concentration of the acid is between about 0.1 M and about 10 M, between about 0.5 M and about 7 M, between about 0.5 M and about 6 M, between about 0.5 M and about 5 M, between about 0.5 M and about 2 M, between about 1 M and about 5 M, or between about 4 M and about 6 M. In one embodiment, including any of the foregoing, the concentration of the HCl is about 0.5 M, about 1 M, about 1.5 M, about 2 M, about 3 M, about 4 M, about 5 M, about 5.5 M, about 6 M, or about 7 M. In one embodiment, including any of the foregoing, the concentration of the acid is about 1 M. In one embodiment, the concentration of the acid is about 5 M.

In one embodiment, the corrosive media comprises hydrochloric acid. In one embodiment, the concentration of the hydrochloric acid is between about 0.1 M and about 10 M, between about 0.5 M and about 7 M, between about 0.5 M and about 5.5 M, between about 0.5 M and about 5 M, between about 0.5 M and about 2 M, between about 1 M and about 5 M, or between about 4 M and about 6 M. In one embodiment, the concentration of the HCl is about 0.5 M, about 1 M, about 1.5 M, about 2 M, about 3 M, about 4 M, about 5 M, about 5.5 M, about 6 M, or about 7 M. In one embodiment, the concentration of the hydrochloric acid is about 1 M. In one embodiment, the concentration of the hydrochloric acid is about 5 M.

In another embodiment, the corrosive media is a different chloride ion-containing media, including, but not limited to, a media containing NaCl.

In another embodiment, the corrosive media comprises brine. In another embodiment, the corrosive media comprises brine and CO₂.

In one embodiment, including any of the foregoing, the concentration of the inhibitor compound of Formula (II) in the corrosive media is between about 1 ppm and about 500 ppm, between about 10 ppm and about 400 ppm, between about 20 ppm and 300 ppm, between about 50 ppm and 250 ppm, between about 100 ppm and 250 ppm, or between about 150 ppm and 250 ppm.

In one embodiment, including any of the foregoing, the concentration of the inhibitor compound of Formula (II) in the corrosive media is between about 1 ppm and about 100 ppm, between about 10 ppm and about 80 ppm, between about 20 ppm and 70 ppm, or between about 20 ppm and 60 ppm.

In one embodiment, including any of the foregoing, the concentration of the inhibitor compound of Formula (II) in the corrosive media is between about 10 ppm and about 200 ppm, between about 10 ppm and about 150 ppm, between about 10 ppm and 120 ppm, or between about 10 ppm and 100 ppm.

In one embodiment, including any of the foregoing, the concentration of the inhibitor compound of Formula (II) in the corrosive media is between about 75 ppm and about 300 ppm, between about 75 ppm and 225 ppm, between about 150 ppm and about 250 ppm, between about 175 ppm and 225 ppm, or between about 75 ppm and 125 ppm.

In one embodiment, including any of the foregoing, the concentration of the inhibitor compound of Formula (II) in the corrosive media is about 10 ppm. In one embodiment, including any of the foregoing, the concentration of the inhibitor compound of Formula (I) in the corrosive media is about 50 ppm. In one embodiment, including any of the foregoing, the concentration of the inhibitor compound of Formula (II) in the corrosive media is about 75 ppm. In one embodiment, including any of the foregoing, the concentration of the inhibitor compound of Formula (II) in the corrosive media is about 100 ppm. In one embodiment, including any of the foregoing, the concentration of the inhibitor compound of Formula (II) in the corrosive media is about 150 ppm. In one embodiment, including any of the foregoing, the concentration of the inhibitor compound of Formula (II) in the corrosive media is about 200 ppm. In one embodiment, including any of the foregoing, the concentration of the inhibitor compound of Formula (II) in the corrosive media is about 250 ppm.

In one embodiment, the corrosive media comprises hydrochloric acid, the concentration of the hydrochloric acid is about 1 M, and the concentration of the inhibitor compound of Formula (II) in the hydrochloric acid is between about 75 pm and 225 ppm. In one embodiment, the corrosive media comprises hydrochloric acid, the concentration of the hydrochloric acid is about 5 M, and the concentration of the inhibitor compound of Formula (I) in the hydrochloric acid is between about 75 pm and 225 ppm.

In one embodiment, including any of the foregoing, the method is conducted at a temperature between about 10° C. to about 80° C. In one embodiment, including any of the foregoing, the method is conducted at a temperature between about 10° C. to about 20° C., between about 10° C. to about 30° C., between about 20° C. to about 40° C., between about 30° C. to about 60° C., between about 50° C. to about 70° C., or between about 60° C. to about 80° C. In one embodiment, including any of the foregoing, including any of the foregoing, the method is conducted at about ambient temperature (23° C.-25° C.). In one embodiment, including any of the foregoing, the method is conducted at about 23° C.

In one embodiment, including any of the foregoing, the method described herein achieves a corrosion inhibition efficiency of greater than 80%, 85%, 90%, 92%, 94%, 95%, 96%, 97%, 98%, or greater. In one embodiment, including any of the foregoing, the method described herein achieves a corrosion inhibition efficiency of greater than 80%. In one embodiment, including any of the foregoing, the method described herein achieves a corrosion inhibition efficiency of greater than 90%. In one embodiment, including any of the foregoing, the method described herein achieves a corrosion inhibition efficiency of greater than 95%. In one embodiment, including any of the foregoing, the method described herein achieves a corrosion inhibition efficiency of greater than 97%.

In one embodiment, including any of the foregoing, the method comprises introducing a compound of Formula (II) wherein X is S. In one embodiment, including any of the foregoing, the method comprises introducing a compound of Formula (II) wherein X is O.

In one embodiment, including any of the foregoing, the method comprises introducing a compound of Formula (II) wherein the compound of Formula (II) is a compound of Formula (II-A) or a salt thereof:

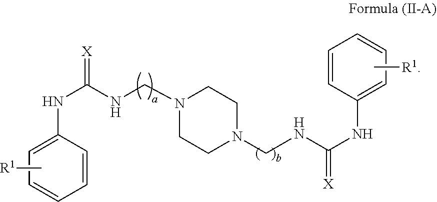

In one embodiment, including any of the foregoing, the method comprises introducing a compound of Formula (II) or Formula (II-A) wherein a is 2. In one embodiment, including any of the foregoing, the method comprises introducing a compound of Formula (II) or Formula (II-A) wherein a is 3. In one embodiment, including any of the foregoing, the method comprises introducing a compound of Formula (II) or Formula (II-A) wherein a is 4. In one embodiment, including any of the foregoing, the method comprises introducing a compound of Formula (II) or Formula (II-A) wherein a is 5. In one embodiment, including any of the foregoing, the method comprises introducing a compound of Formula (II) or Formula (II-A) wherein a is 6. In one embodiment, including any of the foregoing, the method comprises introducing a compound of Formula (II) or Formula (II-A) wherein a is 7. In one embodiment, including any of the foregoing, the method comprises introducing a compound of Formula (II) or Formula (II-A) wherein a is 8. In one embodiment, including any of the foregoing, the method comprises introducing a compound of Formula (II) or Formula (II-A) wherein b is 2. In one embodiment, including any of the foregoing, the method comprises introducing a compound of Formula (II) or Formula (II-A) wherein b is 4. In one embodiment, including any of the foregoing, the method comprises introducing a compound of Formula (II) or Formula (II-A) wherein b is 5. In one embodiment, including any of the foregoing, the method comprises introducing a compound of Formula (II) or Formula (II-A) wherein b is 6. In one embodiment, including any of the foregoing, the method comprises introducing a compound of Formula (II) or Formula (II-A) wherein b is 7. In one embodiment, including any of the foregoing, the method comprises introducing a compound of Formula (II) or Formula (II-A) wherein b is 8. In one embodiment, including any of the foregoing, the method comprises introducing a compound of Formula (II) or Formula (II-A) wherein both a and b are 2. In one embodiment, including any of the foregoing, the method comprises introducing a compound of Formula (II) or Formula (II-A) wherein both a and b are 3. In one embodiment, including any of the foregoing, the method comprises introducing a compound of Formula (II) or Formula (II-A) wherein both a and b are 4. In one embodiment, including any of the foregoing, the method comprises introducing a compound of Formula (II) or Formula (II-A) wherein both a and b are 5. In one embodiment, including any of the foregoing, the method comprises introducing a compound of Formula (II) or Formula (II-A) wherein both a and b are 6. In one embodiment, including any of the foregoing, the method comprises introducing a compound of Formula (II) or Formula (II-A) wherein both a and b are 7. In one embodiment, including any of the foregoing, the method comprises introducing a compound of Formula (II) or Formula (II-A) wherein both a and b are 8.

In one embodiment, including any of the foregoing, the method comprises introducing a compound of Formula (II-A) wherein both R¹ groups are in the ortho-position. In one embodiment, including any of the foregoing, the method comprises introducing a compound of Formula (II-A) wherein both R¹ groups are in the meta-position. In one embodiment, including any of the foregoing, the method comprises introducing a compound of Formula (II-A) wherein both R¹ groups are in the para-position.

In one embodiment, including any of the foregoing, the method comprises introducing a compound of Formula (II-A) wherein R¹ is —NO₂. In one embodiment, including any of the foregoing, the method comprises introducing a compound of Formula (II-A) wherein R¹ is alkyl. In one embodiment, including any of the foregoing, the method comprises introducing a compound of Formula (II-A) wherein R¹ is alkoxy. In one embodiment, including any of the foregoing, the method comprises introducing a compound of Formula (II-A) wherein R¹ is methyl. In one embodiment, including any of the foregoing, the method comprises introducing a compound of Formula (II-A) wherein both R¹ and R² are unsubstituted aryl. In one embodiment, including any of the foregoing, the method comprises introducing a compound of Formula (II-A) wherein both R¹ and R² are aryl substituted with alkyl, alkoxy, —OH, —COOH, —C(O)NH₂, —NH₂, —NHCOOH, —CN, —NO₂, or halogen. In one embodiment, including any of the foregoing, the method comprises introducing a compound of Formula (II-A) wherein both R¹ and R² are unsubstituted heteroaryl. In one embodiment, including any of the foregoing, the method comprises introducing a compound of Formula (II-A) wherein both R¹ and R² are heteroaryl substituted with alkyl, alkoxy, —OH, —COOH, —C(O)NH₂, —NH₂, —NHCOOH, —CN, —NO₂, or halogen. In one embodiment, including any of the foregoing, the method comprises introducing a compound of Formula (II-A) wherein both R¹ and R² are halogen. In one embodiment, including any of the foregoing, the method comprises introducing a compound of Formula (II-A) wherein both R¹ and R² are chloro, bromo, iodo, of fluoro.

In one embodiment, including any of the foregoing, the method comprises introducing a compound of Formula (I). In one embodiment, including any of the foregoing, the method comprises introducing Inhibitor 1. In one embodiment, including any of the foregoing, the method comprises introducing Inhibitor 2.

In one embodiment, the method described herein achieves a corrosion inhibition efficiency of greater than 90% and the method comprises introducing an inhibitor compound of Formula (II) into hydrochloric acid wherein the concentration of the hydrochloric acid is about 1 M and the concentration of the inhibitor compound of Formula (II) in the hydrochloric acid is between about 75 pm and 225 ppm. In a further embodiment, the inhibitor compound of Formula (II) is Inhibitor 1. In a further embodiment, method described herein achieves a corrosion inhibition efficiency of greater than 92%. In a further embodiment, method described herein achieves a corrosion inhibition efficiency of greater than 95%.

In one embodiment, the method described herein achieves a corrosion inhibition efficiency of greater than 90% and the method comprises introducing an inhibitor compound of Formula (II) into hydrochloric acid wherein the concentration of the hydrochloric acid is about 5 M and the concentration of the inhibitor compound of Formula (II) in the hydrochloric acid is between about 150 pm and 225 ppm. In a further embodiment, the inhibitor compound of Formula (II) is Inhibitor 2. In a further embodiment, method described herein achieves a corrosion inhibition efficiency of greater than 95%.

In an alternative embodiment, the method described herein inhibits corrosion of iron alloys such as mild steel, carbon steel, stainless steel. In an alternative embodiment, the method described herein inhibits corrosion of other metals, including, but not limited to zinc and magnesium.

### Synthesis of Compound Inhibitors of Formula (II-A)

Also provided herein is a method for the synthesis of the inhibitor compounds of Formula (II-A) or a salt thereof comprising contacting a bis-amino substituted piperazine of formula 2-1 with a R¹-substituted phenyl compound of formula 1-1 to afford a compound of Formula (II-A) or a salt thereof:

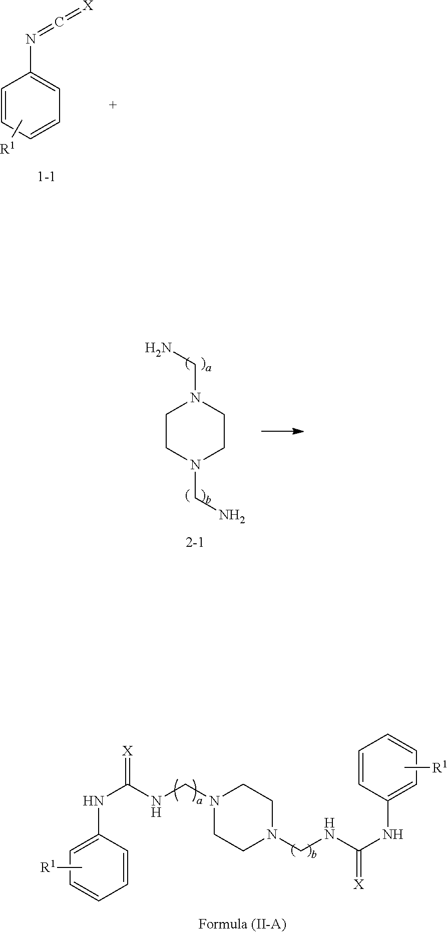

wherein
X is O or S;
R¹ is selected from NO₂, alkyl, aryl, alkoxy, heteroaryl, and halogen wherein the aryl and heteroaryl are optionally substituted with alkyl, alkoxy, —OH, —COOH, —C(O)NH₂, —NH₂, —NHCOOH, —CN, —NO₂, or halogen; and
a and b are independently an integer selected from 2, 3, 4, 5, 6, 7, or 8.

In one embodiment of Formula (1-1), X is S. In one embodiment of Formula (1-1), X is O.

In one embodiment of Formula (1-1), including any of the foregoing, R¹ is in the ortho-position. In one embodiment of Formula (1-1), including any of the foregoing, R¹ is in the meta-position. In one embodiment of Formula (1-1), including any of the foregoing, R¹ is in the para-position.

In one embodiment of Formula (1-1), including any of the foregoing, R¹ is —NO₂. In one embodiment of Formula (1-1), including any of the foregoing, R¹ is alkyl. In one embodiment of Formula (1-1), including any of the foregoing, R¹ is methyl. In one embodiment of Formula (1-1), including any of the foregoing, R¹ is alkoxy. In one embodiment of Formula (1-1), including any of the foregoing, R¹ is unsubstituted aryl. In one embodiment of Formula (1-1), including any of the foregoing, R¹ is aryl substituted with alkyl, alkoxy, —OH, —COOH, —C(O)NH₂, —NH₂, —NHCOOH, —CN, —NO₂, or halogen. In one embodiment of Formula (1-1), including any of the foregoing, R¹ is unsubstituted heteroaryl. In one embodiment of Formula (1-1), including any of the foregoing, R¹ is heteroaryl substituted with alkyl, alkoxy, —OH, —COOH, —C(O)NH₂, —NH₂, —NHCOOH, —CN, —NO₂, or halogen. In one embodiment of Formula (1-1), including any of the foregoing, R¹ is halogen. In one embodiment of Formula (1-1), including any of the foregoing, R¹ is fluoro, bromo, chloro, or iodo.

In one embodiment of Formula (2-1), including any of the foregoing, a is 2. In one embodiment of Formula (2-1), including any of the foregoing, a is 3. In one embodiment of Formula (2-1), including any of the foregoing, a is 4. In one embodiment of Formula (2-1), including any of the foregoing, a is 5. In one embodiment of Formula (2-1), including any of the foregoing, a is 6. In one embodiment of Formula (2-1), including any of the foregoing, a is 7. In one embodiment of Formula (2-1), including any of the foregoing, a is 8. In one embodiment of Formula (2-1), including any of the foregoing, b is 2. In one embodiment of Formula (2-1), including any of the foregoing, b is 3. In one embodiment of Formula (2-1), including any of the foregoing, b is 4. In one embodiment of Formula (2-1), including any of the foregoing, b is 5. In one embodiment of Formula (2-1), including any of the foregoing, b is 6. In one embodiment of Formula (2-1), including any of the foregoing, b is 7. In one embodiment of Formula (2-1), including any of the foregoing, b is 8. In one embodiment of Formula (2-1), including any of the foregoing, both a and b are 2. In one embodiment of Formula (2-1), including any of the foregoing, both a and b are 3. In one embodiment of Formula (2-1), including any of the foregoing, both a and b are 4. In one embodiment of Formula (2-1), including any of the foregoing, both a and b are 5. In one embodiment of Formula (2-1), including any of the foregoing, both a and b are 6. In one embodiment of Formula (2-1), including any of the foregoing, both a and b are 7. In one embodiment of Formula (2-1), including any of the foregoing, both a and b are 8.

In one embodiment, the method comprises contacting a bis-amino substituted piperazine of formula 2-1 with a nitro-substituted phenyl compound of formula 1-1 to afford a compound of Formula (I):

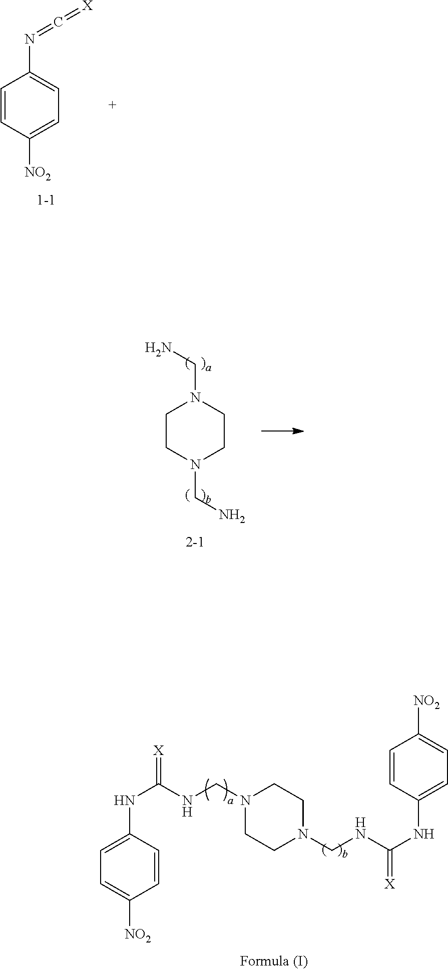

wherein X is O or S; and a and b are independently an integer selected from 2, 3, 4, 5, 6, 7, or 8.

In one embodiment of Formula (1-1), X is S; a and b are both 3; and R¹ is —NO₂ and in the para-position to afford Inhibitor 2. In one embodiment of Formula (1-1), X is O; a and b are both 3; and R¹ is —NO₂ in the para position to afford Inhibitor 1.

In certain embodiments, the synthesis is conducted in one or more organic solvent(s), for example a polar aprotic solvent. In certain embodiments, the polar aprotic solvent is a C₂-C₈ ether, including, but not limited to THF, methyl-t-butyl ether (MTBE), and dioxane. In certain embodiments, the polar aprotic solvent is THF. In certain embodiments, the polar aprotic solvent is a C₃-C₇ ketone, including, but not limited to acetone, propanone, and methyl isobutyl ketone. In certain embodiments, the polar aprotic solvent is a C₃-C₇ nitrile, including, but not limited to acetonitrile (ACN) or propionitrile. In certain embodiments, the polar aprotic solvent is DMF. In certain embodiments, the polar aprotic solvent is DMSO.

Reaction times and reaction conditions (e.g., temperature, atmosphere, etc.) will vary and may be determined by reference to the examples and disclosure provided herein, as well as routine experimentation and consultation of the relevant literature when necessary. In some embodiments, the processes described herein are run under such conditions so as to achieve the desired result.

In one embodiment, the synthesis is conducted at between about −5° C. and 5° C. In one embodiment, the synthesis is conducted at 0° C.

In one embodiment, the reaction is stirred for at least about 12 hours, at least about 10 hours, at least about 8 hours, at least about 4 hours, or at least about 2 hours.

In one embodiment, the reaction is stirred for no more than 30 minutes, no more than 1 hour, no more than 2 hours, no more than 4 hours, no more than 6 hours, no more than 8 hours, no more than 10 hours, no more than 12 hours, no more than 15 hours, or no more than 20 hours.

In one embodiment, the reaction is conducted in open atmosphere and at ambient-light conditions.

In some embodiments, the method may further include purification and isolation steps to remove impurities and/or reactants from the product. Purification of the inhibitor compound of Formula (I) can be obtained by selective crystallization in a solvent or solvent/anti-solvent system, column chromatography, or any method known to skilled chemists that results in such purification.

In certain embodiments, the synthesis is conducted in THF at about 0° C. In certain embodiments, the synthesis is conducted in THF at about 0° C. and the reaction is stirred for about 8 hours.

### EXAMPLES

### Example 1. Synthesis of Inhibitor 1

A solution 4-nitrophenyl isocyanate (1, 2 eq., 1 mmol) in THF (2 mL) was added dropwise to a solution of 1,4-bis(3-aminopropyl) piperazine (2, 1 eq., 1 mmol) in THF (3 mL at 0° C. using an ice bath. The resultant reaction mixture was stirred and allowed to warm gradually to room temperature and stirring continued at this temperature overnight. The resulting white precipitate was filtered and washed by hot acetonitrile and hot ethanol and dried.

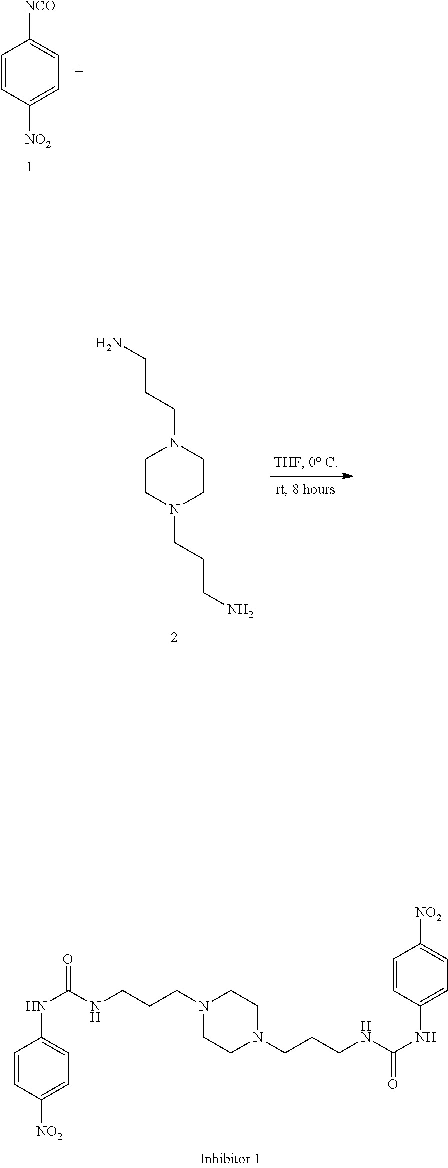

¹H NMR (400 MHz, DMSO) δ 9.33 (s, 2H) Ph-NH, 8.13 (d, J=9.2 Hz, 4H) CHAr, 7.62 (d, J=9.2 Hz, 4H) CHAr, 6.52 (t, J=5.4 Hz, 2H) CH₂NH, 3.12 (dd, J=12.6, 6.5 Hz, 4H) CH₂NH, 2.51-2.30 (m (overlap), 12H), 1.73-1.42 (m, 4H) CH₂CH₂CH₂NH. ¹³CNMR (100 MHz, DMSO) δ 154.90, 147.76, 140.75, 125.60, 117.22, 55.81, 53.30, 38.30, 27.23.

### Example 2. Effect of Inhibitor 1 on Steel Corrosion in 1M HCl

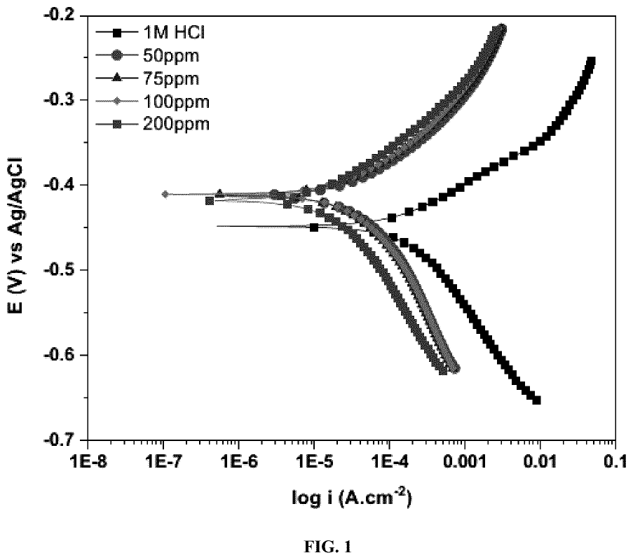

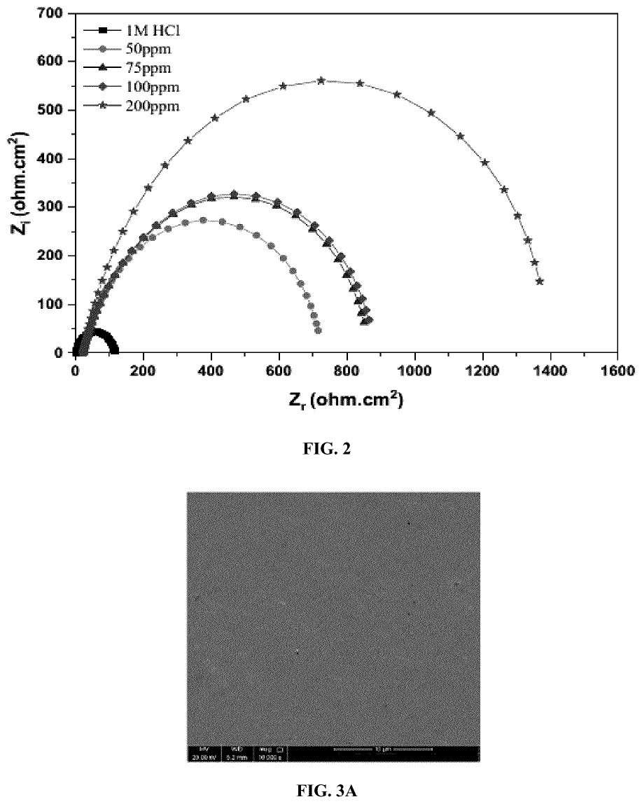

To investigate the effect of Inhibitor 1 on corrosion mechanisms, electrochemical measurements were obtained using Tafel and A.C. impedance experiments. FIGS. 1 and 2 show Tafel and AC impedance curves of carbon steel after adding 50 to 200 ppm of Inhibitor 1 to 1M HCl solution.

The Tafel extrapolation method was used to fit the curves and obtain corrosion parameters. Reduced corrosion rates and current densities with an increase in concentration suggest the formation of a protective layer of Inhibitor 1 on the steel surface. Inhibitor 1 noticeably reduced the corrosion rate of steel by 97% from 71 to 2 mils·year⁻¹ at 200 ppm. The lowest reported current density at 200 ppm concentration was 4.5 μA·cm⁻², three orders of magnitude lower than 155 μA·cm⁻² recorded for blank 1M HCl. Furthermore, the AC impedance demonstrated improved results at higher concentrations as indicated by increased semicircles diameter. The charge transfer resistance reached the maximum of 1430 Ω·cm² at 200 ppm and Inhibitor 1 yielded around 93% efficiency after enhancing it from 116 Ω·cm² for the blank solution. Inhibitor 1 is a highly efficient corrosion inhibitor even at low concentrations; at concentrations of 50 ppm and 75 ppm, it yielded an 82% and 90% efficiency, respectively. The results are shown in Table 1.

TABLE 1
Effect of Inhibitor 1 concentration on current
density and corrosion rate in 1M HCl

| Concentration | Current density | Corrosion rate | % Inhibition |
|---|---|---|---|
| (ppm) | (μA · cm⁻²) | (mils · year⁻¹) | Efficiency |
| Blank | 155 | 71 | — |
| 50 | 29 | 13 | 82 |
| 75 | 15 | 6.8 | 90 |
| 100 | 12 | 5.5 | 92 |
| 200 | 4.5 | 2.0 | 97 |

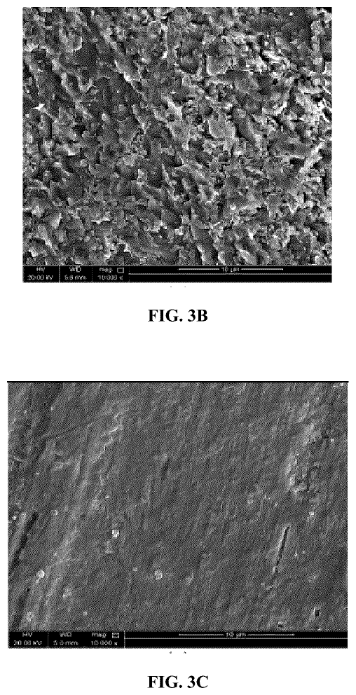

SEM microscopy was used to study the surface topography before and after the corrosion experiments in 1M HCl in the absence and presence of Inhibitor 1. The steel surface (FIG. 3A) before corrosion experiments shows a generally smooth surface, with no significant defects. However, a highly damaged surface was observed in FIG. 3B, with corrosion products covering the surface due to the destructive immersion in the 1M HCl. In contrast, the effect of Inhibitor 1 is shown in FIG. 3C. The surface damage and roughness in FIG. 3C is remarkably reduced and the surface is well protected. This demonstrates the capability of Inhibitor 1 in forming a protective film and effectively retarding corrosion severity.

### Example 3. Predicted Eco-Toxicity Properties of Inhibitor 1

The admetSAR web tool was utilized to assess the eco-toxicity properties of Inhibitor 1. This source predicts properties by relying on a model formulated from 100,000 collected experimental data. The water solubility was estimated from the SwissADME web tool that is based on a model derived from linear regression of 2874 solubility data.

Predicted probabilities of fundamental eco-toxicity descriptors are reported in Table 2. Inhibitor 1 demonstrated a green (safe) prediction for all considered properties. The inhibitor had safe probabilities that reached above 90% from honey bee toxicity and eye corrosion and irritation. This study also showed that the structure had more than a 50% safe impact when exposed to the aquatic living environment. Furthermore, the prediction was about 75% certain that Inhibitor 1 was biodegradable and non-carcinogenic.

TABLE 2
Eco-toxic Properties of Inhibitor 1

| Applicability Domain | In Domain |
|---|---|
| Carcinogenicity | 0.73 |
| Eye Corrosion | 0.98 |
| Eye Irritation | 0.92 |
| Skin Sensitization | 0.83 |
| Honey Bee Toxicity | 0.97 |
| Biodegradation | 0.75 |
| Crustacean Aquatic Toxicity | 0.56 |

### Example 4. Synthesis of Inhibitor 2

A solution of 4-nitrophenyl isothiocyanate (1, 2 eq., 1 mmol) in THF (2 mL) was added dropwise to a solution of 1,4-bis(3-aminopropyl)piperazine (2, 1 eq., 1 mmol) in THF (3 mL) at 0° C. using an ice bath. The resultant reaction mixture was stirred and allowed to warm gradually to room temperature and stirring continued at this temperature overnight. The resulting yellow precipitate was filtered and washed by hot acetonitrile and hot ethanol and finally dried.

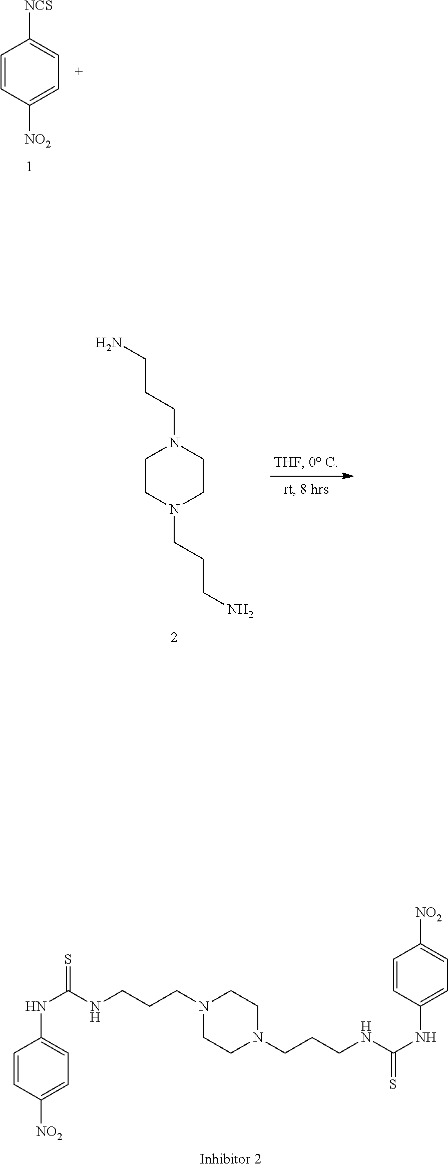

¹H NMR (400 MHz, DMSO) δ 9.33 (s, 2H) Ph-NH, 8.13 (d, J=9.2 Hz, 4H) CHAr, 7.62 (d, J=9.2 Hz, 4H) CHAr, 6.52 (t, J=5.4 Hz, 2H) CH₂NH, 3.12 (dd, J=12.6, 6.5 Hz, 4H) CH₂NH, 2.51-2.30 (m (overlap), 12H), 1.73-1.42 (m, 4H) CH₂CH₂CH₂NH. ¹³CNMR (100 MHz, DMSO) δ 154.90, 147.76, 140.75, 125.60, 117.22, 55.81, 53.30, 38.30, 27.23.

¹H NMR (400 MHz, DMSO) δ 9.90 (s (br), 2H) NH, 8.46 (s (br), 2H) NH, 8.18 (d, J=9.2 Hz, 4H) CHAr, 7.80 (d, J=9.2 Hz, 4H) CHAr, 3.55-3.45 (m, 4H), 2.45-2.20 (m, 12H), 1.85-1.65 (m, 4H). ¹³CNMR (100 MHz, DMSO) δ 180.35, 146.91, 142.13, 125.07, 120.75, 55.94, 53.20, 43.06, 25.68.

### Example 5. Effect of Inhibitor 2 on Steel Corrosion in 5M HCl

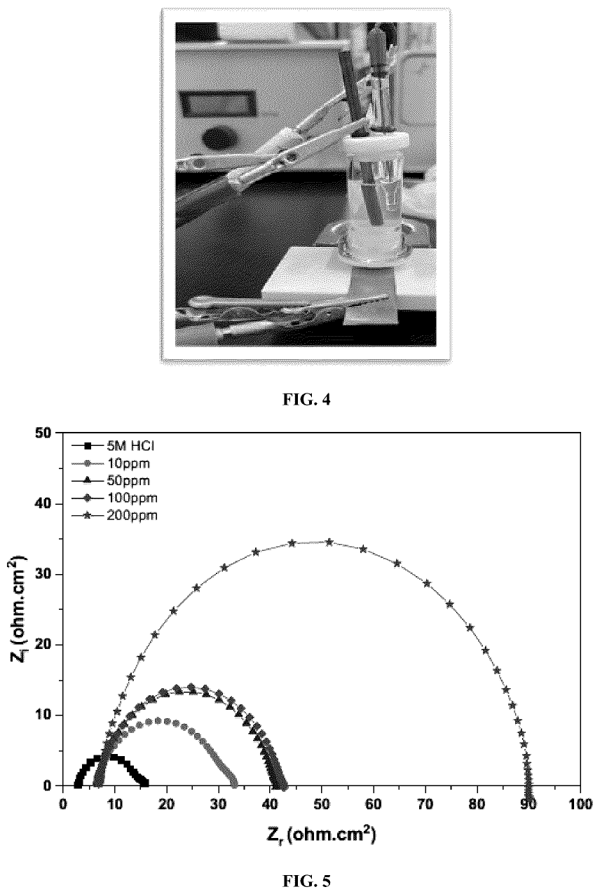

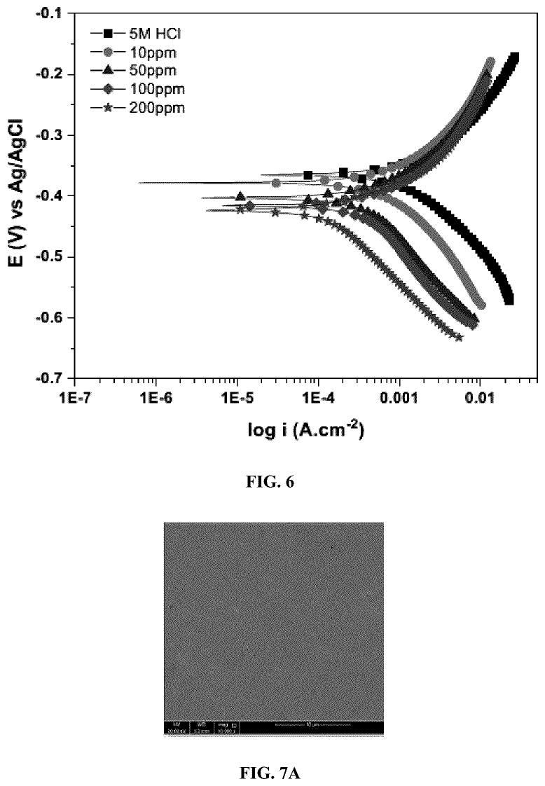

Electrochemical experiments were carried out using the three-electrode cell assembly (FIG. 4). Tafel and AC impedance measurements were obtained to elucidate the corrosion mechanisms and understand the interactions at the solution-surface interface. The effect of adding 10 to 200 ppm Inhibitor 2 on the C-steel corrosion in 5M HCl solution was determined by AC impedance and Tafel curves (FIG. 5 and FIG. 6).

The semicircles of AC impedance curves in FIG. 5 show a remarkably increased diameter upon adding a higher Inhibitor 2 concentration. This demonstrates the adsorption of more molecules onto the C-steel surface, thus improving the inhibitory efficiency (M. S. Hasanin, et al Int. J. Biol. Macromol. 161 (2020) 345-354). The charge transfer resistance (RCT), obtained from fitting the impedance data using an equivalent circuit model, noticeably increased from 12.5 2.cm² for the uninhibited 5M HCl to 83.4 2.cm² for the inhibited solution with 200 ppm of Inhibitor 2. Furthermore, electrochemical parameters describing the kinetics of the corrosion process were acquired following the Tafel extrapolation method to fit the curves of FIG. 6. Results revealed a substantial reduction in the corrosion rate of C-steel in 5M HCl from 846 to 46 mils·year⁻¹ in the presence of 200 ppm Inhibitor 2 (Table 3). Inhibitor 2 successfully achieved 95% efficiency in the highly corrosive condition of 5M HCl at a tolerable quantity of only 200 ppm concentration. The reported current density of 5M HCl was 1850 PA·cm⁻² and was diminished about 18.5-fold to 100 μA·cm⁻² with the inhibitory action of Inhibitor 1.

TABLE 3
Effect of Inhibitor 1 concentration on current
density and corrosion rate in 5M HCl

| Concentration | Current density | Corrosion rate | Inhibition |
|---|---|---|---|
| (ppm) | (μA · cm⁻²) | (mils · year⁻¹) | Efficiency % |
| Blank | 1850 | 846 | — |
| 10 | 787 | 360 | 57 |
| 50 | 429 | 196 | 77 |
| 100 | 209 | 96 | 89 |
| 200 | 100 | 46 | 95 |

The surface morphology of C-steel was investigated using SEM microscopy for the samples before and after corrosion in 5M HCl in the absence and presence of Inhibitor 2.

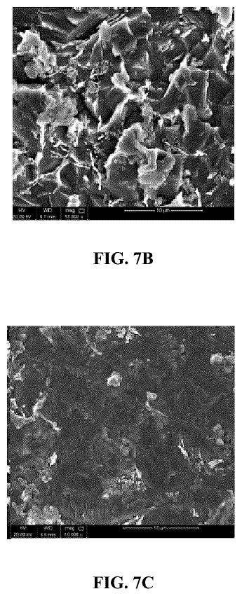

The topography of polished C-steel in FIG. 7A was a very smooth surface with faintly visible scratches after burnishing with abrasive papers. In contrast, after being soaked in the uninhibited 5M HCl solution, the steel exhibited a drastically damaged surface with dense and deep corrosion holes (FIG. 7B). However, the inhibitory influence of Inhibitor 2 is shown in FIG. 7C where the corroded surface roughness was heavily reduced. The SEM micrographs provide evidence of the potential of Inhibitor 2 to interact with a steel surface and form a protective barrier to suppress corrosion.

### Example 6. Predicted Eco-Toxicity Properties of Inhibitor 2

Eco-toxic properties of Inhibitor 2 were predicted using the admetSAR web tool (H. Yang, et al. Bioinformatics. 35 (2019) 1067-1069). The adopted machine-learning model is derived from 100,000 experimental findings. Another web tool, SwissADME a model relying on the regression of 2874 data, was utilized to evaluate the water solubility degree (Swiss Institute of Bioinformatics, SwissADME, (n.d.). http://swissadme.ch/index.php (accessed Dec. 1, 2022).

Table 4 shows probabilities for the essential eco-toxicity descriptors of Inhibitor 2. Inhibitor 2 exhibited safe prediction values for all considered properties and is in the accepted applicability domain. Inhibitor 2 had a 70% probability of being readily biodegradable when exposed to microorganisms in the environment and a 86% probability of possessing non-carcinogenic impacts. Inhibitor 2 also demonstrated more than a 90% safe prediction for honey bee toxicity and eye corrosion or irritation.

The Log(S) for Inhibitor 2 indicated moderate solubility in water (the value for a highly soluble compound must exceed zero).

TABLE 4
Eco-toxic probability properties of Inhibitor 2

| Applicability Domain | In Domain |
|---|---|
| Carcinogenicity | 0.86 |
| Eye Corrosion | 0.98 |
| Eye Irritation | 0.93 |
| Skin Sensitization | 0.81 |
| Honey Bee Toxicity | 0.93 |
| Biodegradation | 0.70 |
| Water Solubility (Log (S)) | −5.05 |

The previous detailed description is of a small number of embodiments for implementing the invention and is not intended to be limiting in scope. One of skill in this art will immediately envisage the methods and variations used to implement this invention in other areas than those described in detail. The following claims set forth a number of the embodiments of the invention disclosed with greater particularity.

What is claimed:

1. A compound of Formula (I): 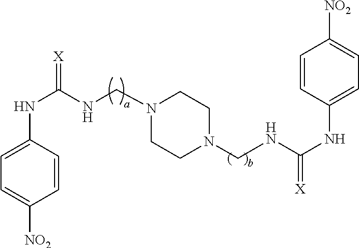
or a salt thereof; wherein X is selected from O and S; and
a and b are independently an integer selected from 2, 3, 4, 5, 6, 7, or 8.

2. The compound of claim 1, where X is O.

3. The compound of claim 1, where X is S.

4. The compound of claim 1, wherein the compound is Inhibitor 1:

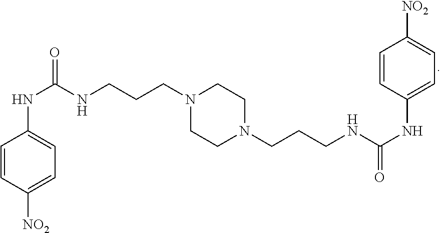 [SMILES: O=C(NCCCN1CCN(CCCNC(=O)Nc2ccc([N+](=O)[O-])cc2)CC1)Nc1ccc([N+](=O)[O-])cc1]

5. The compound of claim 1, wherein the compound is Inhibitor 2:

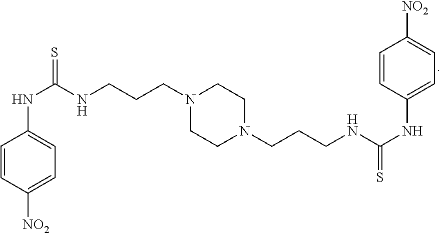 [SMILES: O=[N+]([O-])c1ccc(NC(=S)NCCCN2CCN(CCCNC(=S)Nc3ccc([N+](=O)[O-])cc3)CC2)cc1]

6. A method of inhibiting steel corrosion in the presence of a corrosive media comprising introducing a compound of Formula (II): 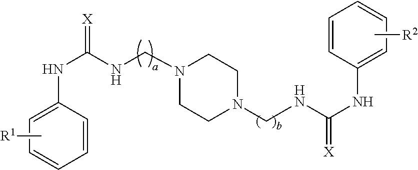
or a salt thereof; wherein
X is selected from O and S;
R¹ and R² are independently selected from NO₂, alkyl, alkoxy, aryl, heteroaryl, and halogen wherein the aryl and heteroaryl are optionally substituted with alkyl, alkoxy, —OH, —COOH, —C(O)NH₂, —NH₂, —NHCOOH, —CN, —NO₂, or halogen; and
a and b are independently an integer selected from 2, 3, 4, 5, 6, 7, and 8; or composition thereof into the corrosive media wherein the steel is in contact with the corrosive media.

7. The method of claim 6, wherein the corrosive media is an aqueous corrosive media.

8. The method of claim 6, wherein the corrosive media is an acid.

9. The method of claim 8, wherein the acid is selected from hydrochloric acid, sulfuric acid, nitric acid, hydrofluoric acid, phosphoric acid, acetic acid, citric acid, and formic acid.

10. The method of claim 9, wherein the acid is hydrochloric acid.

11. The method of claim 6, wherein the concentration of the corrosive media is between about 0.5 M and about 6 M.

12. The method of claim 11, wherein the concentration of the corrosive media is about 1 M.

13. The method of claim 11, wherein the concentration of the corrosive media is about 5 M.

14. The method of claim 6, wherein the concentration of the compound of Formula (I) is between about between about 75 ppm and 225 ppm.

15. The method of claim 14, wherein the concentration of the compound of Formula (I) is about 100 ppm.

16. The method of claim 14, wherein the concentration of the compound of Formula (I) is about 200 ppm.

17. The method of claim 6, wherein the method achieves a corrosion inhibition efficiency of greater than 90%.

18. The method of claim 6, wherein the method achieves a corrosion inhibition efficiency of greater than 95%.

19. The method of claim 6, wherein the compound of Formula (I) is Inhibitor 1:

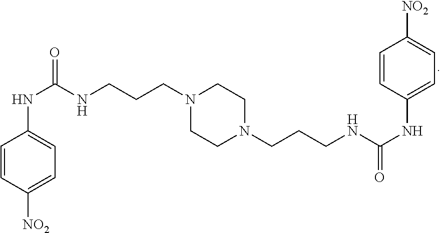 [SMILES: O=C(NCCCN1CCN(CCCNC(=O)Nc2ccc([N+](=O)[O-])cc2)CC1)Nc1ccc([N+](=O)[O-])cc1]

20. The method of claim 6, wherein the compound of Formula (I) is Inhibitor 2:

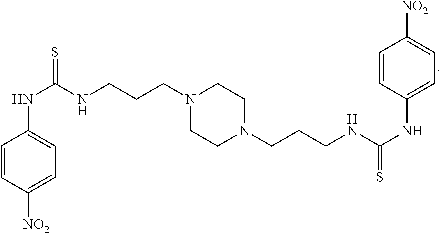 [SMILES: O=[N+]([O-])c1ccc(NC(=S)NCCCN2CCN(CCCNC(=S)Nc3ccc([N+](=O)[O-])cc3)CC2)cc1]

21. A method for the synthesis of a compound of Formula (II-A) or a salt thereof, comprising contacting a bis-amino substituted piperazine of formula 2-1 with a R¹-substituted phenyl compound of formula 1-1 to afford the compound of Formula (II-A): 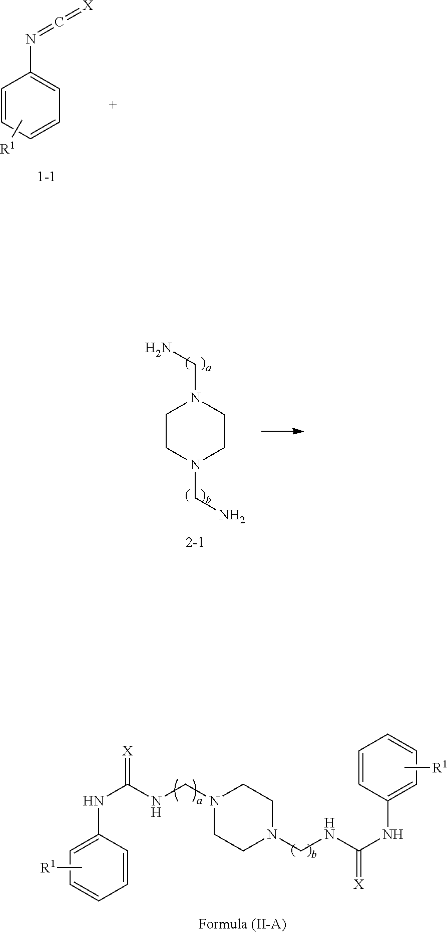
wherein:
X is O or S;
R¹ is selected from NO₂, alkyl, aryl, alkoxy, heteroaryl, and halogen wherein the aryl and heteroaryl are optionally substituted with alkyl, alkoxy, —OH, —COOH, —C(O)NH₂, —NH₂, —NHCOOH, —CN, —NO₂, or halogen; and
a and b are independently an integer selected from 2, 3, 4, 5, 6, 7, or 8.
